(19) United States
(12) Patent Application Publication
(10) Pub. No.: US 2010/0292257 A1
(43) Pub. Date: Nov. 18, 2010

(54) USES OF 2-[PIPERIDINYL] METHYL-2, 3-DIHYDROIMIDAZO [1,2-C] QUINAZOLIN-5 (6H)-ONE FOR PROVIDING AN ANALGESIC EFFECT, ANTI-ALLERGIC EFFECT AND HISTAMINE H1 RECEPTOR ANTAGONISM EFFECT

(75) Inventors: Feng-Nien KO, Taipei (TW); Yuan-Ling Ku, Taipei (TW)

Correspondence Address: BACON & THOMAS, PLLC, 625 SLATERS LANE, FOURTH FLOOR, ALEXANDRIA, VA, 22314-1176, US

(73) Assignee: Medical and Pharmaceutical Industry Technology and Development Center, Taipei Hsien (TW)

(21) Appl. No.: 12/843,364

(22) Filed: Jul. 26, 2010

Related U.S. Application Data
(62) Division of application No. 11/907,853, filed on Oct. 18, 2007.

(30) Foreign Application Priority Data
Sep. 25, 2007 (GB) 0718678.6

(51) Int. Cl. A61K 31/519; A61P 11/06; A61P 37/08

(52) U.S. Cl. 514/267

(57) ABSTRACT

The present invention discloses new uses of 2-[piperidinyl]methyl-2,3-dihydroimidazo[1,2-c]quinazolin-5(6H)-one in providing an analgesic effect in a patient, treating passive cutaneous anaphylaxis in a patient, and in eliciting a histamine H₁ receptor antagonism effect in a patient to treat a disease or disorder, such as allergy.

### CROSS REFERENCES TO THE RELATED APPLICATIONS

This application is a divisional application of pending U.S. patent application Ser. No. 11/907,853, filed Oct. 18, 2007 (of which the entire disclosure of the pending, prior application is hereby incorporated by reference).

### FIELD OF THE INVENTION

The invention of the present application is related to a method of using 2-[piperidinyl]methyl-2,3-dihydroimidazo[1,2-c]quinazolin-5(6H)-one in providing an analgesic effect, anti-allergic effect and a histamine H₁ receptor antagonism effect in a patient.

### BACKGROUND OF THE INVENTION

U.S. Pat. No. 5,158,953 discloses synthesis of a novel series of 2-substituted methyl-2,3-dihydroimidazo[1,2-c]quinazolin-5(6H)-ones (-thiones) compounds, and they are found useful as an active ingredient for the prophylaxis and treatment of hypertension.

U.S. Pat. No. 5,340,814 and U.S. Pat. No. 5,512,677 disclose a novel series of 3-substituted methyl-2,3-dihydroimidazo[1,2-c]quinazoline-5(6H)-ones (-thiones) compounds. These compounds are found useful as an active ingredient for the treatment of hypertension and dysuria.

U.S. Pat. No. 5,932,584 discloses novel optically active 3-substituted methyl-5-methylthio-2,3-dihydroimidazo[1,2-c]quinazoline (I) and 3-substituted methyl-2,3-dihydroimidazo[1,2-c]quinazolin-5(6H)-one (II). These compounds are found useful as an active ingredient for the treatment of hypertension and dysuria.

U.S. Pat. No. 6,946,470B2 discloses a new use of 2-[piperidinyl]methyl-2,3-dihydroimidazo[1,2-c]quinazolin-5(6H)-one in treating psychosis in a patient.

Heretofore, the 2-[piperidinyl]methyl-2,3-dihydroimidazo[1,2-c]quinazolin-5(6H)-one compounds have not been found other pharmaceutical activity in addition to as an active ingredient for the treatment of hypertension, dysuria, and psychosis in patient.

### SUMMARY OF THE INVENTION

An objective of the present invention is to provide a new use of 2-[piperidinyl]methyl-2,3-dihydroimidazo[1,2-c]quinazolin-5(6H)-one in providing an analgesic effect in a patient.

Another objective of the present invention is to provide a new use of 2-[piperidinyl]methyl-2,3-dihydroimidazo[1,2-c]quinazolin-5(6H)-one in treating passive cutaneous anaphylaxis in a patient.

Still another objective of the present invention is to provide a new use of 2-[piperidinyl]methyl-2,3-dihydroimidazo[1,2-c]quinazolin-5(6H)-one in eliciting a histamine H₁ receptor antagonism effect in a patient to treat a disease or disorder.

Accordingly, the present invention provides a method of providing an analgesic effect to a patient in need thereof comprising administering to the patient an analgesia therapeutically effective amount of 2-[piperidinyl]methyl-2,3-dihydroimidazo[1,2-c]quinazolin-5(6H)-one having the following formula (I) or a pharmaceutically acceptable salt thereof:

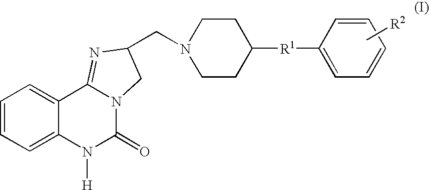

wherein R¹ is C1-C6 alkylene, carbonyl, C1-C6 alkylene carbonyl or carbonyloxy; and R² is hydrogen, C1-C6 alkyl, C1-C6 alkoxy or halogen.

The present invention also provides a method of treating a passive cutaneous anaphylaxis in a patient comprising administering to the patient a therapeutically effective amount of 2-[piperidinyl]methyl-2,3-dihydroimidazo[1,2-c]quinazolin-5(6H)-one having the formula (I) recited above or a pharmaceutically acceptable salt thereof.

The present invention also provides a method of eliciting a histamine H₁ receptor antagonism effect in a patient to treat a disease or disorder such as allergic rhinitis or asthma, which comprises administering to the patient a therapeutically effective amount of 2-[piperidinyl]methyl-2,3-dihydroimidazo[1,2-c]quinazolin-5(6H)-one having the formula (I) recited above or a pharmaceutically acceptable salt thereof as a histamine H₁ receptor antagonist.

Preferably, R¹ is methylene or carbonyl, and more preferably is carbonyl.

Preferably, R² is hydrogen or halogen, more preferably is halogen, and most preferably is fluorine.

Preferably, said 2-[piperidinyl]methyl-2,3-dihydroimidazo[1,2-c]quinazolin-5(6H)-one is 2-[1-(4-p-fluorobenzoyl)piperidinyl]methyl-2,3-dihydroimidazo[1,2-c]-quinazolin-5(6H)-one.

Preferably, said 2-[piperidinyl]methyl-2,3-dihydroimidazo[1,2-c]quinazolin-5(6H)-one or a pharmaceutically acceptable salt thereof is orally administered.

### DETAILED DESCRIPTION OF THE INVENTION

2-[Piperidinyl]methyl-2,3-dihydroimidazo[1,2-c]quinazolin-5(6H)-ones were synthesized according to the method disclosed in U.S. Pat. No. 5,858,953, the details of which are incorporated herein by reference. Phenylquinone (PQ)-induced writhing assay and acetic acid-induced writhing assay were conducted to evaluate these compounds as a potential analgesic drug.

Passive cutaneous anaphylaxis assay was conducted to evaluate these compounds as a potential anti-allergic drug.

Histamine H₁ antagonism assay was conducted to evaluate these compounds as a potential histamine H₁ antagonist.

The invention is further described by means of example, but not in any limitative sense.

Percentages and other amounts referred to in this specification are by weight unless indicated otherwise. Percentages are selected from any ranges used to total 100%.

### Phenylquinone (PQ)-Induced Writhing Assay

Vehicle (2% Tween 80, 10 ml/kg), PDC-130 or aspirin was orally administered to a group of eight CD-1 (Crl.) derived male mice weighing 24±2 g. One hour later, phenylquinone (2 mg/kg) was given by intraperitoneal injection. Writhes of animals was observed and recorded during a period of time from the 5^{th} to 10^{th} minutes after PQ administration, [Reference: Siegmund, E, Cadmus, R. and Lu, G. A method for evaluating both non-narcotic and narcotic analgesics. Proc. Soc. Exp. Biol. Med. 952: 729-731, 1957.]

TABLE 1
Inhibition on Phenylquinone-induced Writhing by PDC-130

| Treatment | Dose | Number of Writhing |
|---|---|---|
| Vehicle | 10 ml/kg | 15.1 ± 1.4 |
| PDC-130* | 1 mg/kg | 2.8 ± 1.2*** |
| | 0.3 mg/kg | 5.3 ± 1.7*** |
| | 0.1 mg/kg | 6.4 ± 1.6*** |
| | 0.03 mg/kg | 12.1 ± 1.6 |
| Aspirin | 100 mg/kg | 5.5 ± 1.5*** |

*PDC-130: 2-[1-(4-p-fluorobenzoyl)piperidinyl]methyl-2,3-dihydroimidazo[1,2-c]-quinazolin-5(6H)-one (Example 15 in U.S. Pat. No. 5,158,953)
***p < 0.001 as compared with the vehicle control.

As shown in Table 1 the extremely significant reduction in the number of writhes in PDC-130 treated animals as compared with the vehicle control group indicates a possible analgesic activity.

### Acetic Acid-Induced Writhing Assay

Vehicle (2% Tween 80, 10 ml/kg), PDC-130 or aspirin (100 mg/kg) was administered orally to groups of eight CD-1 (Crl.) derived male mice, weighing 24±2 g, 1 hour before intraperitoneal injection of acetic acid (0.5%, 20 ml/kg). The number of writhes of animals was observed and recorded during a period of time from the 5^{th} to the 10^{th} minutes period after acetic acid administration. [Reference: Inoue. K., Motonaga, A. and Nishimura, T. Mechanism of antiinflammatory action of etodolac. Arzneim-Forsch./Drug Res. 41: 235-239, 1991.]

TABLE 2
Inhibition on Acetic Acid-induced Writhing by PDC-130

| Treatment | Dose | Number of Writhing |
|---|---|---|
| Vehicle | 10 ml/kg | 12.4 ± 1.5 |
| PDC-130* | 1 mg/kg | 2.1 ± 1.1*** |
| | 0.3 mg/kg | 5.3 ± 1.1*** |
| | 0.1 mg/kg | 5.0 ± 1.0*** |
| | 0.03 mg/kg | 12.1 ± 1.6 |
| Aspirin | 100 mg/kg | 2.4 ± 1.0*** |

*PDC-130: 2-[1-(4-p-fluorobenzoyl)piperidinyl]methyl-2,3-dihydroimidazo[1,2-c]-quinazolin-5(6H)-one (Example 15 in U.S. Pat. No. 5,158,953)
***p < 0.001 as compared with the vehicle control.

As shown in Table 2 the extremely significant reduction in the number of writhes in PDC-130 treated animals as compared with the vehicle control group indicates a possible analgesic activity.

### Passive Cutaneous Anaphylaxis

A group of five Wistar derived male rats weighing 80±20 g was passively sensitized 16 hours earlier by intradermal injection of reaginic antiovalbumin serum (0.5 ml) on two spots of the dorsal surface. Vehicle (2% Tween 80), PDC-130 or cyproheptadine was orally administered. Within one hour after administration of the above substances, the animals were challenged intravenously with a mixture of ovalbumin (1 mg) and Evans Blue dye (5 mg) and sacrificed 30 minutes later. The two wheal diameters were measured for each animal and scored as follows:
Score 0: diameter<0.05 cm
Score 1: diameter 0.05-0.20 cm
Score 2: diameter 0.2-0.4 cm
Score 3: diameter 0.4-0.6 cm
Score 4: diameter 0.6-0.8 cm
Score 5: diameter>0.8 cm

Maximum possible score for each animal total 5×2=10. [Reference: Goose, J. and Blair, A. M. J. N. Passive cutaneous anaphylaxis in the rat, induced with two homologous reagin-like antibodies and its specific inhibition with disodium chromoglycate. Immunology 16: 749-760, 1969.]

TABLE 3
Inhibitory Effects of PDC-130 on Passive Cutaneous Anaphylaxis

| Treatment | Dose | Total Score | Inhibition** (%) |
|---|---|---|---|
| Vehicle | 10 ml/kg | 50 | — |
| PDC-130* | 30 mg/kg | 10 | 80 |
| | 10 mg/kg | 10 | 80 |
| | 3 mg/kg | 16 | 68 |
| Cyproheptadine | 1 mg/kg | 14 | 72 |

*PDC-130: 2-[1-(4-p-fluorobenzoyl)piperidinyl]methyl-2,3-dihydroimidazo[1,2-c]-quinazolin-5(6H)-one (Example 15 in U.S. Pat. No. 5,158,953)
**Inhibition percentage of the resulting passive cutaneous anaphylaxis blue colored wheal was calculated as follows: (Total score of vehicle group − Total score of tested group)/(Total score of vehicle group) × 100%

(Total score of vehicle group−Total score of tested group)/(Total score of vehicle group)×100%

It can be seen from Table 3 that the good inhibition percentages of the resulting passive cutaneous anaphylaxis blue colored wheal in the PDC-130 groups indicate a possible anti-allergic activity.

### Histamine H₁ Antagonism

Vehicle (2% Tween 80), PDC-130 or cyproheptadine was orally administered to a group of five Wistar derived male rats weighing 80±20 g. After one hour, animals were injected with Evans Blue dye (5 mg/0.5 ml/rat) intravenously and immediately challenged with two intradermal injection of histamine (each 30 μg/0.05 ml). The animals were sacrificed 30 minutes later. The two wheal diameters were then measured for each animal and scored as follows:
Score 0: diameter<0.05 cm
Score 1: diameter 0.05-0.20 cm
Score 2: diameter 0.2-0.4 cm
Score 3: diameter 0.4-0.6 cm
Score 4: diameter 0.6-0.8 cm
Score 5: diameter>0.8 cm

Maximum possible score for each animal total 5×2=10.

TABLE 4
Antagonism of Histamine H₁ Receptor by PDC-130

| Treatment | Dose | Total Score | Inhibition** (%) |
|---|---|---|---|
| Vehicle | 10 ml/kg | 50 | — |
| PDC-130* | 30 mg/kg | 10 | 80 |
| | 10 mg/kg | 14 | 72 |
| | 3 mg/kg | 20 | 60 |
| Cyproheptadine | 1 mg/kg | 18 | 64 |

*PDC-130: 2-[1-(4-p-fluorobenzoyl)piperidinyl]methyl-2,3-dihydroimidazo[1,2-c]-quinazolin-5(6H)-one (Example 15 in U.S. Pat. No. 5,158,953)
**Inhibition percentage of the resulting histamine-induced blue colored wheal was calculated as follows: (Total score of vehicle group − Total score of tested group)/(Total score of vehicle group) × 100%

It can be seen from Table 4 that the good inhibition percentages of the histamine-induced blue colored wheal in the PDC-130 groups indicates a possible histamine H₁ receptor antagonism.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

What is claimed is:

1. A method of eliciting a histamine H₁ receptor antagonism effect in a patient to treat a disease or disorder comprising administering to the patient a therapeutically effective amount of 2-[piperidinyl]methyl-2,3-dihydroimidazo[1,2-c]quinazolin-5(6H)-one having the following formula or a pharmaceutically acceptable salt thereof as a histamine H₁ receptor antagonist: 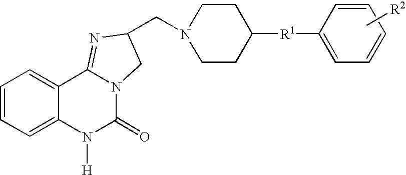
wherein R¹ is C1-C6 alkylene, carbonyl, C1-C6 alkylene carbonyl or carbonyloxy; and R² is hydrogen, C1-C6 alkyl, C1-C6 alkoxy or halogen.

2. The method according to claim 1, wherein said disease or disorder is allergic rhinitis or asthma.

3. The method according to claim 1, wherein R¹ is methylene or carbonyl.

4. The method according to claim 3, wherein R¹ is carbonyl.

5. The method according to claim 1, wherein R² is hydrogen or halogen.

6. The method according to claim 3, wherein R² is hydrogen or halogen.

7. The method according to claim 4, wherein R² is halogen.

8. The method according to claim 7, wherein R² is fluorine.

9. The method according to claim 8, wherein said 2-[piperidinyl]methyl-2,3-dihydroimidazo[1,2-c]quinazolin-5(6H)-one is 2-[1-(4-p-fluorobenzoyl)piperidinyl]methyl-2,3-dihydroimidazo[1,2-c]-quinazolin-5(6H)-one.

10. A method of treating a passive cutaneous anaphylaxis in a patient comprising administering to the patient a therapeutically effective amount of 2-[piperidinyl]methyl-2,3-dihydroimidazo[1,2-c]quinazolin-5(6H)-one having the following formula or a pharmaceutically acceptable salt thereof: 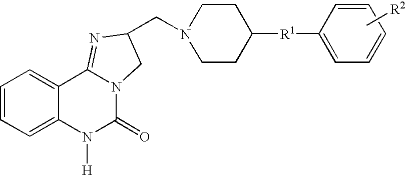
wherein R¹ is C1-C6 alkylene, carbonyl, C1-C6 alkylene carbonyl or carbonyloxy; and R² is hydrogen, C1-C6 alkyl, C1-C6 alkoxy or halogen.

11. The method according to claim 10, wherein R¹ is methylene or carbonyl.

12. The method according to claim 11, wherein R¹ is carbonyl.

13. The method according to claim 10, wherein R² is hydrogen or halogen.

14. The method according to claim 11, wherein R² is hydrogen or halogen.

15. The method according to claim 12, wherein R² is halogen.

16. The method according to claim 15, wherein R² is fluorine.

17. The method according to claim 16, wherein said 2-[piperidinyl]methyl-2,3-dihydroimidazo[1,2-c]quinazolin-5(6H)-one is 2-[1-(4-p-fluorobenzoyl)piperidinyl]methyl-2,3-dihydroimidazo[1,2-c]-quinazolin-5(6H)-one.
